(19) United States
(12) Patent Application Publication
(10) Pub. No.: US 2026/0091189 A1
(43) Pub. Date: Apr. 2, 2026

(54) TRACHEOSTOMY TUBE WITH DECANNULATION-RESISTANT SECUREMENT DEVICE

(71) Applicant: Christiana Care Health System, Inc., Wilmington, DE (US)

(72) Inventors: Michael Szeliga, Newark, DE (US); Hayley Sweetser, Hockessin, DE (US)

(21) Appl. No.: 19/321,574

(22) Filed: Sep. 8, 2025

Related U.S. Application Data
(60) Provisional application No. 63/700,054, filed on Sep. 27, 2024.

(51) Int. Cl. A61M 16/04; A61M 16/00

(52) U.S. Cl. CPC A61M 16/0465; A61M 16/0003; A61M 16/044; A61M 2205/0205; A61M 2209/06; A61M 2209/088; A61M 2210/1032

(57) ABSTRACT

A removal-resistant securement device suitable for use with a conventional tracheostomy tube and other devices, and a tracheostomy tube including the securement device are provided. The securement device has a body including an enlarged shield portion, and laterally disposed strap portions. The body is elongated sufficiently to pass over the neck/body part, through the tracheostomy tube/device, and to allow the strap portions to wrap back around the neck and over the shield. End portions of the strap(s) include mechanical or other fasteners for securely mating the ends of the strap portions, preferably at a point overlying a nape of the neck for a tracheostomy device. The shield portion may be larger in size than the straps to reduce pressure on the skin, and to facilitate arrangement of the fastener(s) over the shield portion, so that it can act as a physical barrier between the fastener(s) and the skin.

### CROSS-REFERENCE TO RELATED APPLICATION

This application claims the benefit under 35 U.S. C. § 119(e) of U.S. Provisional Ser. No. 63/700,054 , filed Sep. 27, 2024, the entire disclosure of which is hereby incorporated by reference.

### FIELD OF THE INVENTION

The present invention relates generally to tracheotomy procedures and tracheostomy breathing tubes, and more particularly, to a tracheostomy tube with a decannulation-resistant securement device configured to prevent unintended or unauthorized (self-initiated) decannulation resulting in a loss of a person's airway as the result of removal of the tracheostomy tube from the person's trachea.

### DISCUSSION OF RELATED ART

People may experience a need for breathing assistance as a result of an accident or a health/medical issue. Mechanical ventilator machines are configured to provide a supply of air, via a ventilator tube, to provide such breathing assistance.

In certain circumstances, a tracheotomy procedure is performed to provide the person with a tracheostomy tube. As well known in the art, the tracheotomy procedure involves creating a hole in the person's neck, through the person's skin and bodily tissues, so that the tracheostomy tube can be inserted through the hole and into the person's trachea/windpipe. When in use with a ventilator machine, an outer portion of the tracheostomy tube is connected to the ventilator tube of the ventilator machine, and an inner portion of the tracheostomy tube resides within the person's trachea to maintain an open airway and deliver the air supplied from the ventilator machine to the person's lungs via the tracheostomy tube and trachea, as well known in the art. As patients become more stable, patients will be weaned from ventilator support to only supplemental oxygen and humidification, and in this case, the ventilator tube is disconnected, and the outer portion of the tracheostomy tube is not connected to anything. In a typical use case, the tracheostomy tube is essential or critical to maintaining the patient's airway and/or proper ventilation.

Disruption, partial dislodgement and/or complete removal (i.e., decannulation), referred to collectively herein as “dislodgement”, of the tracheostomy tube, whether deliberately or accidentally, can be a life-threatening event. Unauthorized/unsupervised self-decannulation can be a particular concern for persons with cognitive impairment. Unauthorized/unsupervised dislodgement generally requires an immediate response and intervention from the healthcare team to restore cannulation and/or otherwise restore the patient's airway and/or proper ventilation to prevent death by asphyxiation, etc.

Presently, dislodgement is generally identified during routine rounds by the person's care team (which may be asynchronous with and long after a dislodgement event), or by reporting of the event by a patient's visitor, or if the patient is being remotely monitored, by changes in blood oxygen saturation and/or heart rate as detected by purpose-specific sensors and associated monitoring equipment, which can only indirectly suggest that there might be a tracheostomy dislodgement issue. Further, abnormal blood oxygen and/or heart rate do not actually/directly detect decannulation/dislodgement, but rather detects results that may occur asynchronous after a time period after such dislodgement, and thus cannot serve to ensure swift rectification of any breathing issue in the event of dislodgement. Accordingly, though maintenance of the airway is critical to patient health, detection of a tracheostomy tube dislodgement may be delayed relative to occurrence of the dislodgement, which jeopardies the patient's health, and is therefore undesirable.

Accordingly, to prevent decannulation, after the tracheostomy tube has been properly inserted into the person's trachea/windpipe, the tube is typically secured to the person's body/neck by a strap. In one common example, a flange 126 of the tracheostomy tube 100 defines a pair of openings 140a, 140b in the neck plate/flange 126 to provide a pair of opposed mounts 165a, 165b for a securement strap 150. The strap 150 is passed through the openings 140, 140b for securing purposes. In one exemplary known embodiment, the strap 150 has a neck band body 152 that is soft or padded for patient comfort when applied against the skin, and a pair of ends 154a, 154b that are passed through the flange 126. Further, each end 154a, 154b has complementary fields of hook fastener and loop fastener of a hook-and-loop fasteners system, such as that sold commercially as Velcro fastener. For example, each end 154a, 154b. may include a landing field 156a, 156b of hook fastener and a distal tail field 158a, 158b of loop fastener that can be passed through an opening 140a, 140b, and be pressed against, and thereby mated to, the landing field to secure the strap around the person's neck, and the tracheostomy tube 100 to the patient's body, as shown in FIG. 2.

In another common example, the strap 150 is provided with a neck band body 160 in the nature of a tie that includes a first portion 162 having a first end 164a secured to the flange 126 through the first opening 140a and a second end 166a opposite the first end, and a second portion 162b having a first end 164b secured to the flange 126 through the second opening 140b and a second end 166b opposite the first end, such that the second ends 166a, 166b are free ends that can be looped once around the person's neck and be tied at the back of the next in a loose knot 170 to secure the strap 150 around the person's neck, and the tracheostomy tube 100 to the patient's body, as shown in FIG. 3.

However, these straps are easily unfastened, and are not designed to prevent someone from loosening them and decannulating. More particularly, the knot of the tie is easy to locate and can be easily undone by the patient, and similarly, the free ends of the hook-and-loop embodiment are typically located at the front of the neck where they may be easily located and unsecured, either accidentally or intentionally by the patient (who is unauthorized to perform decannulation).

What is needed is an improved approach for securing a tracheostomy tube that can deter or prevent decannulation, either accidentally or unintentionally, and particularly, by the patient.

### SUMMARY

The present invention provides a decannulation-resistant securement device suitable for use with a conventional tracheostomy tube and other person-worn devices, and a novel tracheostomy tube including a decannulation-resistant securement device that can deter or prevent decannulation, either accidentally or unintentionally, and particularly, by the patient.

The decannulation-resistant securement device has an elongated body configured to safely secure the tracheostomy tube structure to/around a person's neck in a manner that is resistant to accidental and/or easy dislodgement/decannulation. More particularly, the body is elongated to be sufficient in length to span a region of the person's neck, between opposed mounts of the neck plate/flange of the tracheostomy tube, etc. In certain embodiments, the body is elongated sufficiently to be looped twice around the person's neck (or other body part, between the opposed mounts) and have a remaining fastening portion used to fasten the free ends to each other to secure the body/strap around the person's neck, etc. and the tracheostomy tube/device to the patient's body.

The body defines a shield portion and a pair of laterally disposed strap portions. The shield may be centrally located on the body, but in any event is positioned so that the ends of the strap portions can be brought together over the shield.

The strap portions include fasteners, such as snaps, hooks, or other mechanical or other interference-type fasteners for secure engagement. When the fasteners are fastened in a position overlying the shield.

The shield may be worn over a posterior portion of the wearer's neck, where it is difficult for the person/wearer to reach and unfasten the device. The strap portions are sufficient in length to extend along the sides/front of the wearer's neck, to loop through secure points of the tracheostomy tube, etc., and to double back along the sides/rear of the wearer's neck to overlie the shield. The shield portion has a greater height than the straps to aid in positioning the fasteners over the shield, so the shield acts as a physical barrier/buffer to reduce discomfort and/or abrasions to the skin that could result from direct contact with the fasteners, and/or to reduce the pressure on the skin underlying the shield due to the relatively larger area of the shield.

### BRIEF DESCRIPTION OF THE FIGURES

An understanding of the following description will be facilitated by reference to the attached drawings, in which:

FIG. 1 is a side view of a person in a hospital bed connected to a mechanical ventilator machine to receive breathing assistance/air via an exemplary tracheostomy tube having a decannulation-resistant securement device in accordance with the present invention;

FIG. 2 is a perspective view of a first exemplary tracheostomy tube including a first securing strap in accordance with the prior art;

FIG. 3 is a perspective view of a second exemplary tracheostomy tube including a second securing strap in accordance with the prior art;

FIG. 4 is a plan view of an exemplary decannulation-resistant securement strap in accordance with an exemplary embodiment of the present invention;

FIG. 5 is a perspective view of decannulation-resistant securement device of FIG. 4 shown in use with an exemplary tracheostomy tube in accordance with an exemplary embodiment of the present invention;

FIG. 6 is a rear perspective view of a tracheostomy tube with the decannulation-resistant securement device of FIG. 5 shown in use and worn about a person's neck in accordance with an exemplary embodiment of the present invention; and

FIG. 7 is a perspective view of a tracheostomy kit including a co-packaged tracheostomy tube and the decannulation-resistant securement device of FIG. 5.

### DETAILED DESCRIPTION

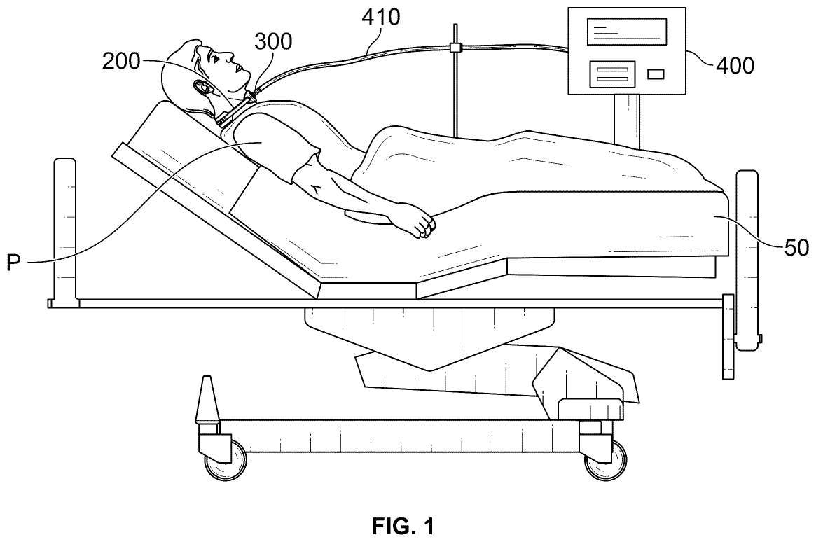

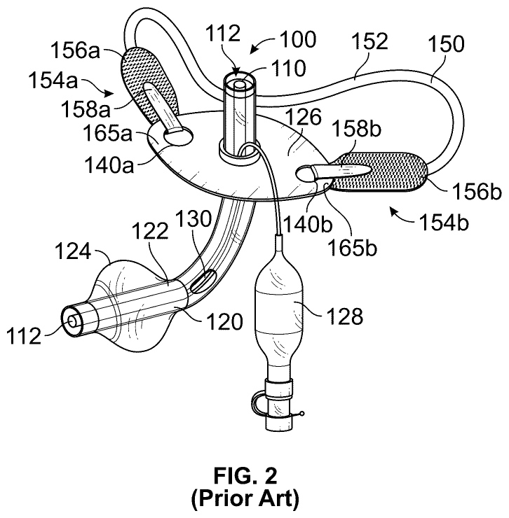

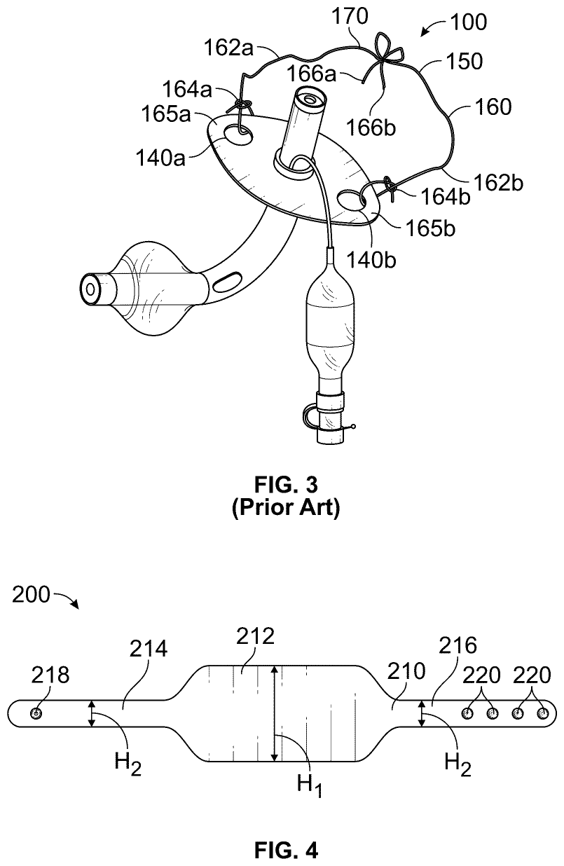

FIG. 1 is a side view of a person in a hospital bed having a tracheostomy tube 300 in accordance with the present invention connected to a mechanical ventilator machine 400 to receive breathing assistance/air via the tracheostomy tube.

The tracheostomy tube 300 may have largely (or entirely) conventional tracheostomy tube structure. However, unlike convention tracheostomy tubes, a tracheostomy tube in accordance with the present invention includes a novel decannulation-resistant securement device 200 in accordance with the present invention, as best shown in FIGS. 4-6.

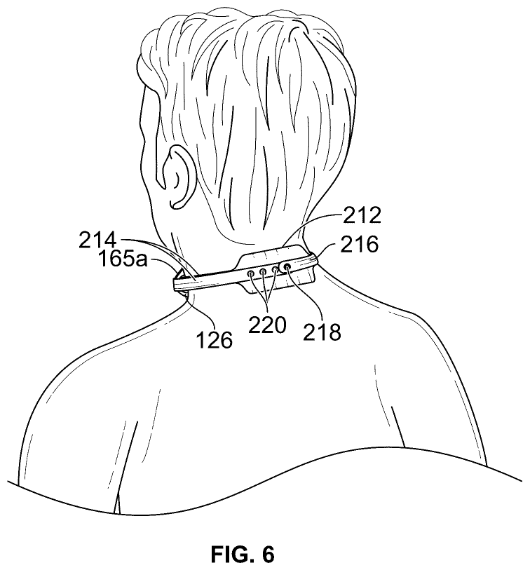

As shown in FIGS. 4-6, the decannulation-resistant securement device 200 has an elongated body 210 configured to safely secure the tracheostomy tube structure to/around a person's neck in a manner that is resistant to accidental and/or easy dislodgement/decannulation. More particularly, the body 210 is elongated to be sufficient in length to span a region of the person's neck, between opposed mounts 165a, 165b of the neck plate/flange 126 of the tracheostomy tube 100. In certain embodiments, the body is elongated sufficiently to be looped twice around the person's neck (between the opposed mounts 165a, 165b) and have a remaining fastening portion used to fasten the free ends to each other to secure the strap 150 around the person's neck, and the tracheostomy tube 100 to the patient's body, as will be appreciated from FIG. 6. Accordingly, in certain embodiments, the body 210 may have a length of two times or more a distance around a human neck/neck size to which it is intended to be fastened. Accordingly, the decannulation-resistant securement device 200 is configured as a single continuous body member that spans both opposed mounts 165a, 165b to provide a securing mounting, in contrast to common tracheostomy devices.

The body 210 of the decannulation-resistant securement device 200 defines a central shield portion 212 and a pair of laterally disposed strap portions 214, 216. The body 210 is elongated so that in use, as shown in FIG. 6, the shield portion 212 may be worn over a posterior portion of the wearers neck, while the strap portions 214, 216 are sufficient in length extend along the sides/front of the wearer's neck, to loop through secure points of the tracheostomy tube, and to double back along the sides/rear of the wearer's neck, as best shown in FIG. 6. The shield portion 212 has a maximum height H₁ greater than a maximum height H₂ of the laterally disposed strap portions 214, 216, as shown in FIG. 4. In certain embodiments, the height of a predominant portion of the shield is greater than the maximum heigh of the laterally disposed strap potions 214, 216, or at least greater than a height of the portions of the laterally disposed strap portions 214, 216 that will overlie the shield 212 when the tracheostomy tube 300 and decannulation-resistant securement device 200 are secured around a person's neck.

The relatively greater height of the shield 212 provides a relatively larger surface area that reduces pressure per unit area on the bodily tissue overlain by the shield 212. Additionally, providing a shield 212 having a surface area greater than that of the fastening portions of the strap portions 214, 216 helps to ensure that any fasteners of the end portions of the strap portions 214, 216 will be positioned over the shield portion when the tracheostomy tube 300 and decannulation-resistant securement device 200 are secured around a person's neck, such that the shield 212 will act as a physical barrier separating buttons, snaps or other fasteners of the strap portions 214, 216 from bodily tissue of the neck, to reduce any likelihood of injury, chafing, discomfort, or neck tissue degradation.

The strap portions 214, 216 may be relatively narrow to facilitate passage through the openings 140a, 150b/opposed mounts 165a, 165b of the neck plate 126 of the tracheostomy tube 300, and the central shield portion 212 may be relatively broad to act as a shield and physical barrier to the fasteners securing the strap portions 214, 216 to each other, as further discussed below.

Strap portion 214 includes a first portion of a fastener, and strap portion 216 includes a second, complementary, portion of the fastener, so that the fastener portions can be mated to secure the strap portions 214, 216 to each other, and the tracheostomy device 100 to the securement device 200 and neck of the wearer. Preferably, the fasteners are selected to be securable to each other to provide a secure, mechanically-interlocking fit, in contrast to a string tie or hook-and-loop-type fastener.

In the exemplary embodiment shown, strap portion 214 includes at least one snap stud (male portion) 218 of a snap fastener (such as an MRI-compatible snap fastener of a type commonly used on hospital gowns), and strap portion 216 includes at least one snap stud receiver (female portion) 220 of the snap fastener. Providing more than one snap stud and/or snap stud receiver facilitates tight/secure fastening of the decannulation-resistance securement device around necks of various sizes at a point overlying a posterior portion of the wearer's neck (to reduce/eliminate the likelihood of dislodgement/release of the securement device due to confusion, discomfort, or accidental impact, or even purposeful dislodgement when the snap (or other fastener) is provided such that it fastens securely enough such that it is not easy to remove easily, even using both hands, which is made even more difficult by the placement at the back of the neck), and overlying the central shield portion 212 of the securement device, which provides a physical buffer/shield between the snap/fastener and the patient's skin, to avoid skin abrasion/tissue damage.

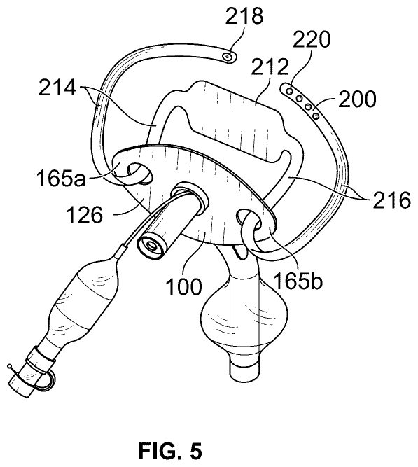

Alternative mechanical fasteners using a structural interference and/or interference fit include matable interlocking metal/rigid hooks or clips, such as an open box clasp, buckle clasp, push button clasp, or slide and lock clasp, or any other suitable structure known in the art. Accordingly, the decannulation-resistant securement device 200 avoids the need for Velcro straps to secure the plate that are easily accessible by confused, disoriented patients, and easily unfastened, and instead using strong, secure mechanical fasteners, and provides a strap/body with a shield 212 for physically buffering the mechanical fasteners from the skin of the neck, as will be best appreciated from FIGS. 5 and 6.

Further, in certain embodiments the body 210 is generally symmetrical, with the shield 212 centrally located along its length, so that the strap portions 214, 216 extend from the ends, such that the shield will overlie the back of the neck, and the ends of the strap portions 214 ,216 will be fastened to each other at the back of the neck, when the tracheostomy tube is secured to the neck. Preferably, the decannulation-resistant securement device 200 is configured to be secured/coupled at the back of the neck (when worn in the typical fashion) to limit the patient's accessibility to the decannulation-resistant securement device, and thereby prevent accidental or intentionally uncoupling that could lead to decannulation. This avoids providing fasteners at the front of the neck/neck plate 126 where the person can easily reach and possibly unfasten them, resulting in dislodgement/decannulation. FIG. 5 shows the attachment to the neck plate 126 via a snap fastener, and that the decannulation-resistant securement device 200 doubles back on itself and is fastened behind the neck.

The decannulation-resistant securement device body 210 may be formed of a single piece of fabric or other material to allow for safe and secure securement of the tracheostomy tube to the neck without the risk of easy dislodgement.

Further, the decannulation-resistant securement device 200 may include a body made of a lightweight, breathable material that reduces friction and shear forces on the skin, and thus prevents damage to the skin and is comfortable to wear. The body may be made of a lightweight, breathable material, such as a knitted polyester textile dressing. This material reduces friction and shear forces on the skin, and thus prevents damage to the skin and is comfortable to wear. The breathability of this fabric can reduce the risk of moisture-associated skin damage common in patients with tracheostomies due to sputum accumulation. The addition of silver to the dressing will add an antimicrobial property that can prevent fungal and bacterial skin infections due to moisture accumulation. The fabric may be configured to allow a slight stretch, allowing it to be applied snugly to the patient's neck without discomfort. The material may be antimicrobial (e.g., include silver threads and/or coatings) and be moisture wicking to minimize skin breakdown risks. Preferably, a material is selected to have a high degree of resistance to ripping/tearing by hand.

Further still, the decannulation-resistant securement device 200 may be constructed with a body that is designed to be frangible, and thus to break, in the event of pulling or excessive force applied to the decannulation-resistant securement device 200 or tracheostomy tube 300, to avoid a ligature risk when high levels of force are applied. By way of example, the device, or body 210, maybe constructed of a material designed to yield and/or break under an specified load, and/or may be configured with narrowed, thinned, pre-cut, or similar defined frangible areas designed to cause the device 200 or yield and/or break under the specified load, to act somewhat like a mechanical fuse calibrated to break before ligature-related damage to the wearer. Alternatively, the snap/fastener may be configured to break or release easily when both sides of the decannulation-resistant securement device are pulled with a predetermined degree of force, to avoid a ligature risk when undesirably high levels of force are applied to similarly act as a mechanical fuse to avoid a ligature risk.

It should be noted that the securement device may be used in other contexts to secure other body-mounted devices to other body parts, and that in such circumstances, the securement device may be removal-resistant, and not decannulation resistant, though the structures are the same or similar.

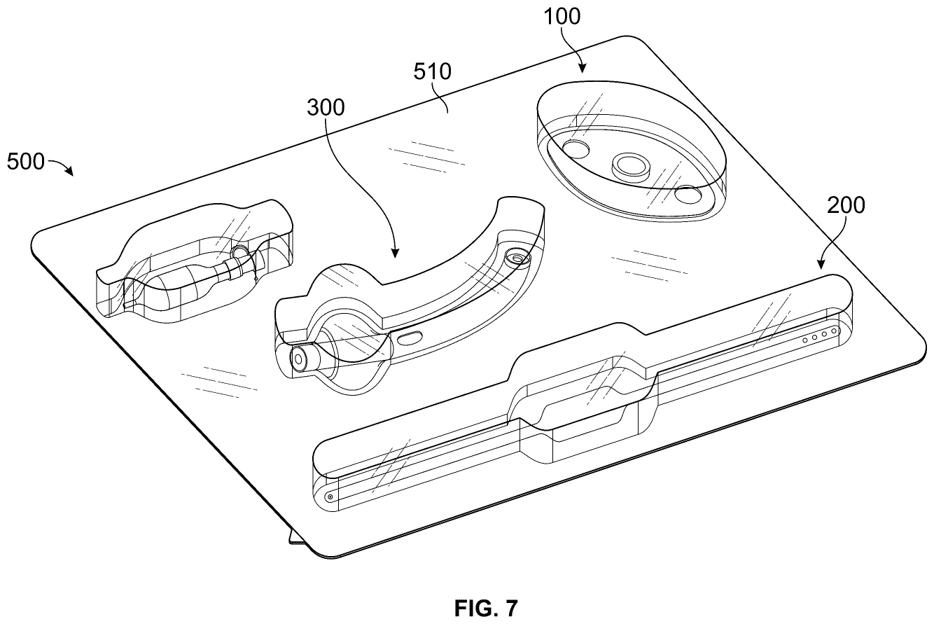

In use, a securement device 200 in accordance with the present invention may be sold as a standalone item for fitment to a conventional/commercially available tracheostomy tube 100 or other body-mounted device, e.g., one obtained separately. Alternatively, a novel tracheostomy tube 300 having a generally-conventional structure may be sold/distributed/received/originally equipped with a decannulation-resistant securement device 200 co-packaged with or already positioned through the both openings 140a, 140b and/or the opposed mounts 165a, 165b of the tracheostomy tube's face plate 126, such that the tracheostomy tube and decannulation-resistant securement device 200 are obtained together, e.g., in a common package. Accordingly, in some embodiments, a tracheostomy tube 100/300 and securement device 200 may be co-packaged in a common package body 510 that encloses both the tracheostomy tube and the securement device, to form a tracheostomy kit 500, as shown in FIG. 7.

A person may be prepared for intubation with tracheostomy tube, and the tracheostomy tube may be inserted into the person's trachea, using the conventional techniques.

By way of example, the tracheostomy tube may then be secured to/about the person's neck in an operative position by threading one end of the device 200 through both openings 140a, 140b or otherwise connecting the decannulation-resistant securement device 200 to the opposed mounts 165a, 165b of the tracheostomy tube's face plate 126, e.g., after the tracheostomy tube has been inserted into the person's trachea using conventional procedures. The decannulation-resistant securement device 200 can be positioned so that the distal ends of the laterally disposed strap portions 214,216 (namely, the portions bearing the fastener(s)) are positioned to overlie the shield 212 of the device 200. Depending upon the position of the shield 212 and/or lengths of the laterally disposed strap portions 214, 216, the shield 212 is preferably positioned at, or near, the back (nape) of the neck. The laterally disposed strap portions 214, 216 can then be pulled snugly through the openings 140a, 140b, and around the neck, to cause the fasteners (e.g., a snap stud fastener 218 and one of the stud receiver fasteners 220 to align) to be securely (e.g., mechanically) mated in a snug position around the neck. In this manner, two layers of the decannulation-resistant securement device 200 overlie the tissue of the neck (between the face plate's 126 opposed mounts 165a, 165b), and the fasteners are positioned to overlie the shield 212, and preferably with the shield positioned over the nape of the neck. In this manner, the fasteners are physically buffered from the skin of the neck to avoid abrasion/chafing/tissue damage, and the fastener(s) are located in a location that is relatively difficult for the person to reach, so as to uncouple. In this manner, and with the use of secure fasteners, accidental and/or unauthorized uncoupling of the fasteners, and resulting decannulation, is relatively unlikely.

If needed, the device can be removed (e.g., by an authorized person at an appropriate time) by decoupling the fasteners and loosening or removing the decannulation-resistant securement device 200, before or after removing the tracheostomy tube from the person's trachea/windpipe.

Accordingly, the present invention may provide a sturdier, cooling, foam/microfoam, antimicrobial tracheostomy tie that is compatible with current tracheostomy collars on the market to allow for a more comfortable, safer product that can reduce adverse patient harm events. The sturdier, more durable material allows for a decreased likelihood of non-purposeful, unsecurement and decannulation, and the cooling foam/microfoam antimicrobial technology allows for a more comfortable fit, and decreases the likelihood of skin breakdown and site infections. The design enhances securement of person-mounted devices in a comfortable, safe fashion. The decannulation-resistant securement device may be configured to fasten any other medical devices, and thus is usable with devices other than tracheostomy tubes, as will be appreciated by those skilled in the art.

Accordingly, the present invention provides a securement device that can better prevent removal (particularly unintentional and/or unauthorized removal) by deterring patients from manipulating the securement device, while also avoiding ligature marks and/or strangulation, and while avoiding moisture wicking into the strap material that can lead to skin damage.

Although the present invention is described above primarily in relation to a hospital setting, it should be noted that the device of the present invention may also be used in long-term care and skilled nursing facilities, in patient homes, and may be particularly advantageous for use with pediatric patients.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A securement device configured to secure to a body part a body-mounted device having a pair of opposed mounts, the securement device comprising:
a body defining:
a shield portion; and
a pair of opposed strap portions, each of the pair of opposed strap portions extending laterally from opposed ends of the shield portion; and
a fastener disposed on at least one of the pair of opposed strap portions, the fastener being operable to secure end portions of the pair of opposed strap portions to each other;
wherein the body is elongated to permit each of the pair of opposed strap portions to pass though a respective one of the pair of opposed mounts while the body-mounted device is worn against the body part, and while the shield portion is worn against the body part to trap the body part between the body-mounted device and the shield portion, and to permit the fastener to secure the end portions of the pair of opposed strap portions to each other at a location overlying the shield portion to secure the body-mounted device to the body part.

2. The securement device of claim 1, wherein the body has a length of at least twice a perimeter of the body part.

3. The securement device of claim 1, wherein the shield portion is disposed centrally to a length of the body.

4. The securement device of claim 3, wherein the shield portion is disposed at a point spanning a middle of a length of the body.

5. The securement device of claim 3, wherein the shield portion is disposed to be positioned opposite the body part from the body-mounted device when worn on the body part.

6. The securement device of claim 1, wherein an end portion of each of the pair of opposed strap portions has a height less than that of the shield portion.

7. The securement device of claim 1, wherein the fastener comprises a first fastener portion and a complementary second fastener portion matable to the first fastener portion, the first fastener portion being disposed on a first of the pair of opposed strap portions, the second fastener portion being disposed on a second of the pair of opposed strap portions.

8. The securement device of claim 7, wherein at least one of the first and the second of the pair of opposed strap portions comprises a plurality of first or second fastener portions.

9. The securement device of claim 1, wherein one of the first and second fastener portions comprises a snap stud, and another one of the first and second fastener portions comprises a snap stud receiver.

10. The securement device of claim 1, wherein the fastener is a mechanical fastener fastenable by way of a structural interference or interference fit.

11. The securement device of claim 1, wherein the body is constructed of a fabric.

12. The securement device of claim 1, wherein the body is constructed of an antimicrobial material.

13. The securement device of claim 1, wherein the body is constructed of a breathable material.

14. The securement device of claim 1, wherein the body is constructed to have an antimicrobial property.

15. The securement device of claim 1, wherein the first and second fastener portions are configured to decouple in response to application of a predetermined load, the predetermined load being selected to avoid a ligature risk.

16. The securement device of claim 1, wherein the body is configured to yield or break in response to application of a predetermined load, the predetermined load being selected to avoid a ligature risk.

17. A tracheostomy tube comprising:
a neck plate having a pair of opposed mounts;
a first sidewall supported on the neck plate and defining a first cannula configured to admit passage of a flow of air;
a second sidewall supported on the neck plate and defining a second cannula in fluid communication with an inflatable cuff driven by a pilot balloon; and
a securement device configured to secure the neck plate and sidewalls to a person's neck, the securement device comprising:
a body defining:
a shield portion; and
a pair of opposed strap portions, each of the pair of opposed strap portions extending laterally from opposed ends of the shield portion; and
a fastener disposed on at least one of the pair of opposed strap portions, the fastener being operable to secure end portions of the pair of opposed strap portions to each other;
wherein the body is elongated to permit each of the pair of opposed strap portions to pass through a respective one of the pair of opposed mounts while the neck plate is worn against an anterior portion of the neck, and while the shield portion is worn against another portion of the neck to trap the neck between the neck plate and body, and to permit the fastener to secure the end portions of the pair of opposed strap portions to each other at a location overlying the shield portion to secure the neck plate and sidewalls to the neck.

18. The tracheostomy tube of claim 17, wherein the body has a length of at least twice a perimeter of the neck.

19. The tracheostomy tube of claim 17, wherein the shield is at a point spanning a middle of a length of the body.

20. The tracheostomy tube of claim 17, wherein an end portion of each of the pair of opposed strap portions has a height less than that of the shield portion.

21. The tracheostomy tube of claim 17, wherein the fastener comprises a first fastener portion and a complementary second fastener portion matable to the first fastener portion, the first fastener portion being disposed on a first of the pair of opposed strap portions, the second fastener portion being disposed on a second of the pair of opposed strap portions.

22. The tracheostomy tube of claim 21, wherein at least one of the first and the second of the pair of opposed strap portions comprises a plurality of first or second fastener portions.

23. The tracheostomy tube of claim 17, wherein one the first and second fastener portions comprises a snap stud, and another one of the first and second fastener portions comprises a snap stud receiver.

24. The tracheostomy tube of claim 17, wherein the body is constructed of a breathable material.

25. The tracheostomy tube of claim 17, wherein the body is constructed to have an antimicrobial property.

26. The tracheostomy tube of claim 17, wherein the first and second fastener portions are configured to decouple in response to application of a predetermined load, the predetermined load being selected to avoid a ligature risk.

27. The tracheostomy tube of claim 17, wherein the body is configured to yield or break in response to application of a predetermined load, the predetermined load being selected to avoid a ligature risk.

28. A tracheostomy kit comprising:
a tracheostomy tube comprising:
a neck plate having a pair of opposed mounts;
a first sidewall supported on the neck plate and defining a first cannula configured to admit passage of a flow of air; and
a second sidewall supported on the neck plate and defining a second cannula in fluid communication with an inflatable cuff driven by a pilot balloon;
a securement device configured to secure the neck plate and sidewalls to a person's neck, the securement device comprising:
a body defining:
a shield portion; and
a pair of opposed strap portions, each of the pair of opposed strap portions extending laterally from opposed ends of the shield portion; and
a fastener disposed on at least one of the pair of opposed strap portions, the fastener being operable to secure end portions of the pair of opposed strap portions to each other;
wherein the body is elongated to permit each of the pair of opposed strap portions to pass through a respective one of the pair of opposed mounts while the neck plate is worn against an anterior portion of the neck, and while the shield portion is worn against another portion of the neck to trap the neck between the neck plate and body, and to permit the fastener to secure the end portions of the pair of opposed strap portions to each other at a location overlying the shield portion to secure the neck plate and sidewalls to the neck; and
a package body enclosing the tracheostomy tube and the securement device.
